(19) United States
(12) Patent Application Publication
(10) Pub. No.: US 2013/0303829 A1
(43) Pub. Date: Nov. 14, 2013

(54) Balancing acupuncture points using resonant signals

(71) Applicant: Edward Ludwig Blendermann, Palm City, FL (US)

(72) Inventor: Edward Ludwig Blendermann, Palm City, FL (US)

(21) Appl. No.: 13/506,737

(22) Filed: May 14, 2012

(51) Int. Cl. A61N 2/08

(52) U.S. Cl. 600/15

(57) ABSTRACT

A method of balancing acupuncture points by wearing a holding device that includes a waterproof container containing substances that give off resonant frequencies that have been found to balance acupuncture points allowing the wearer to receive therapy continually by wearing such a holding device. The holding device may be in the form of a locket, jewelry, patch with a medical adhesive, belt, pedal device, or incorporated into clothing and veterinary apparel.

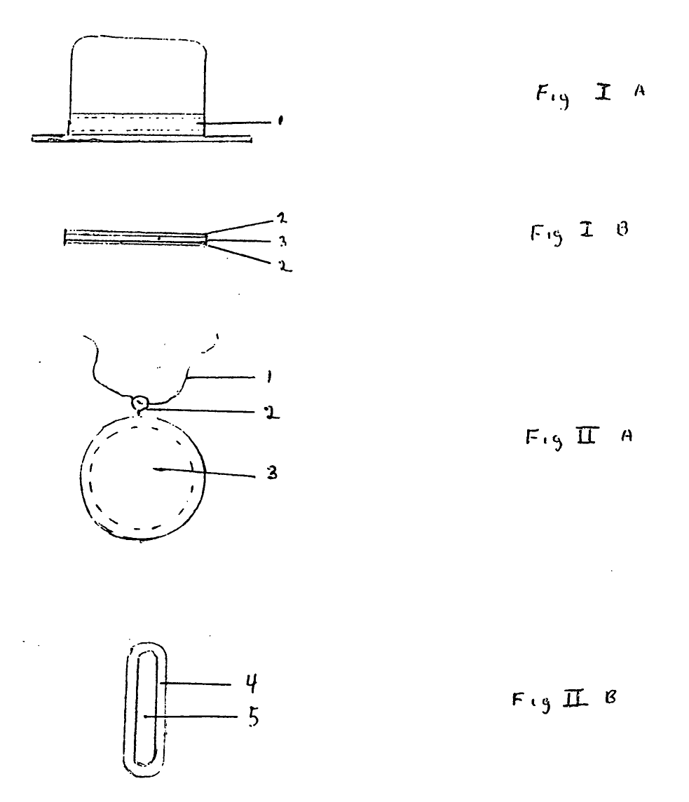

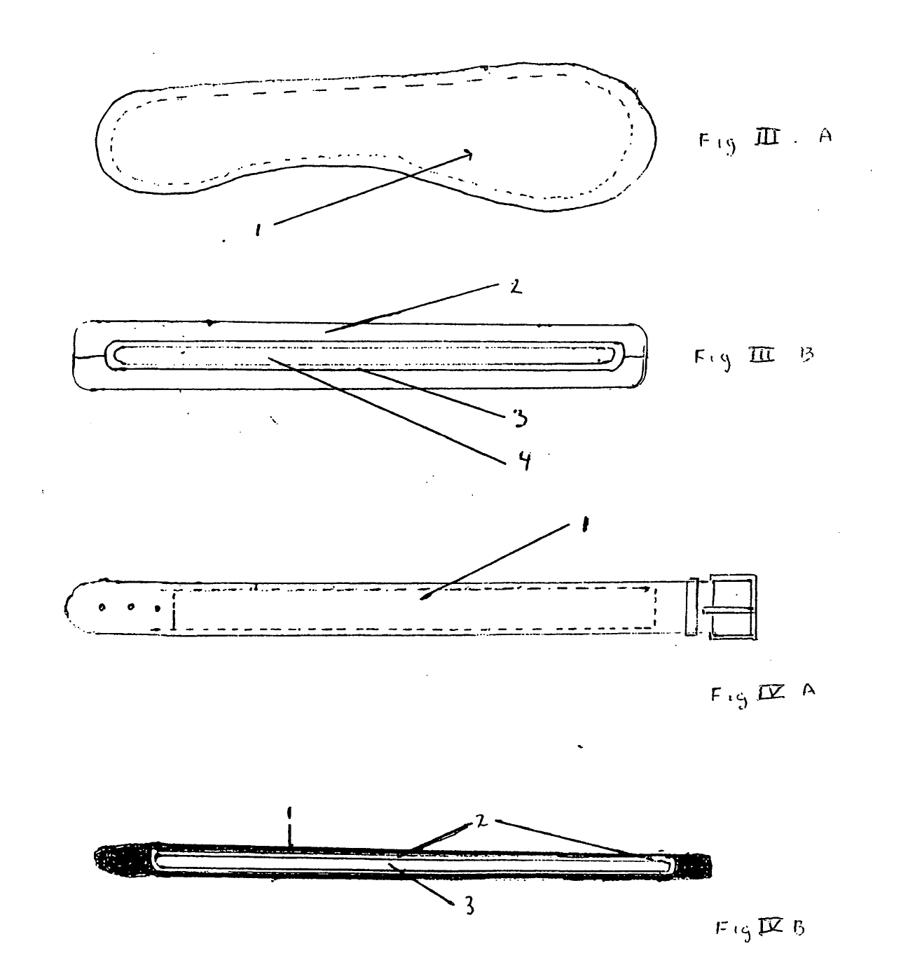

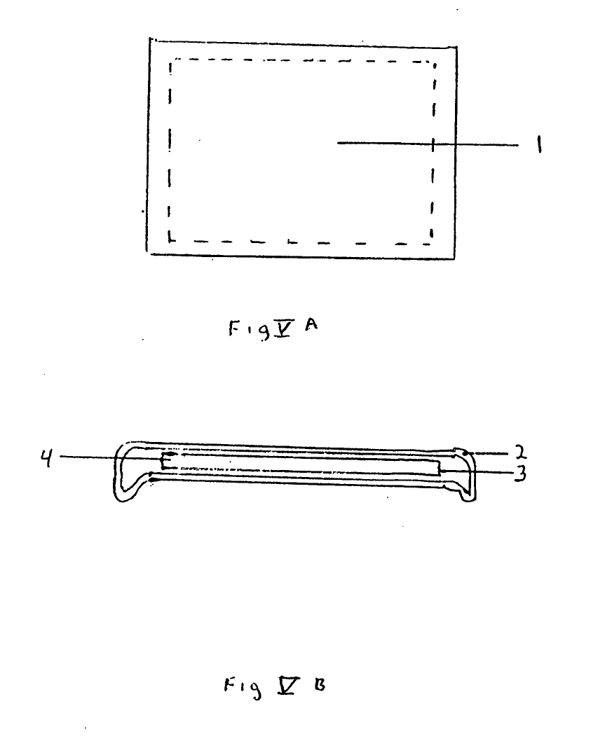

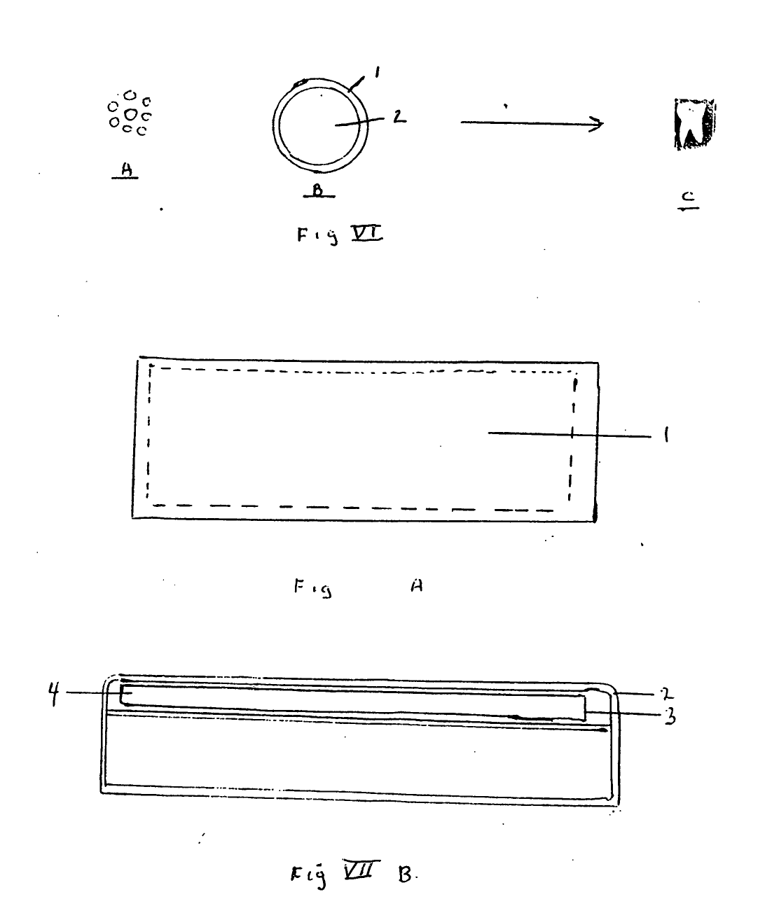

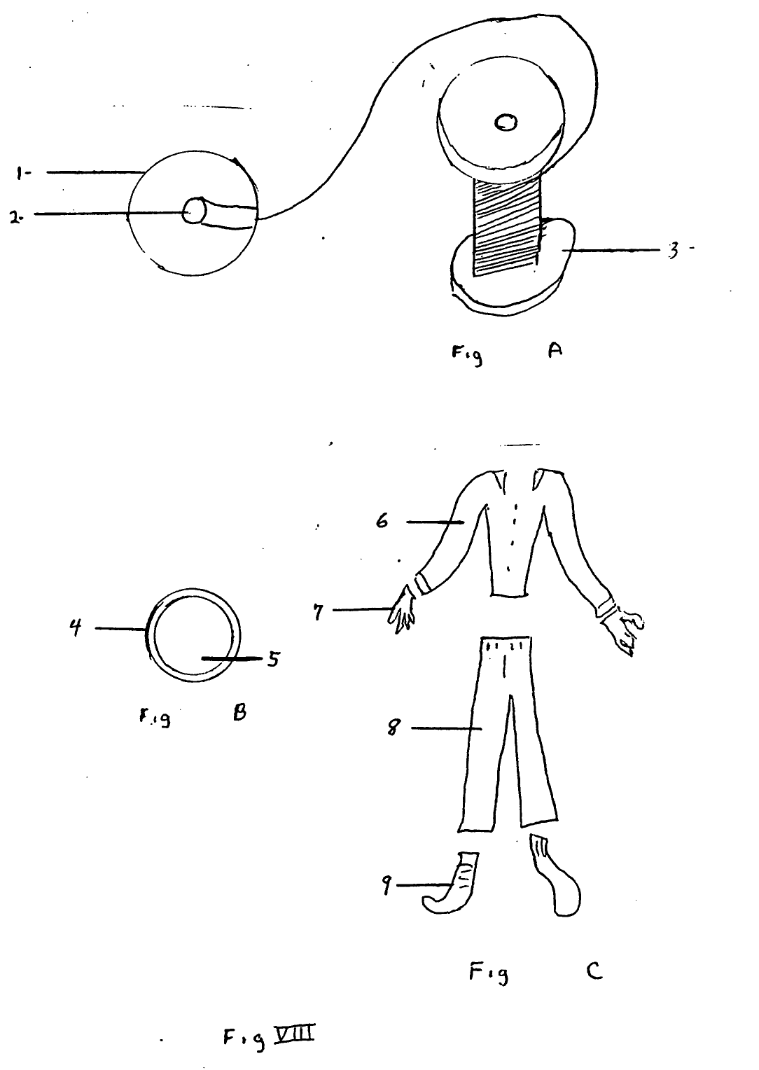

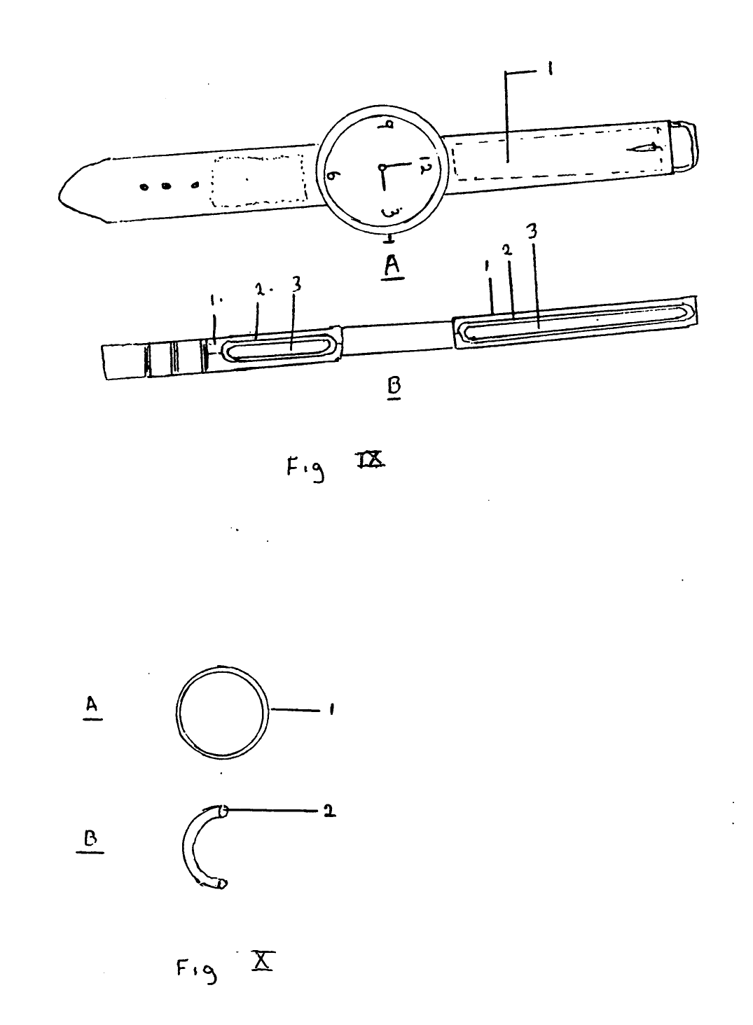

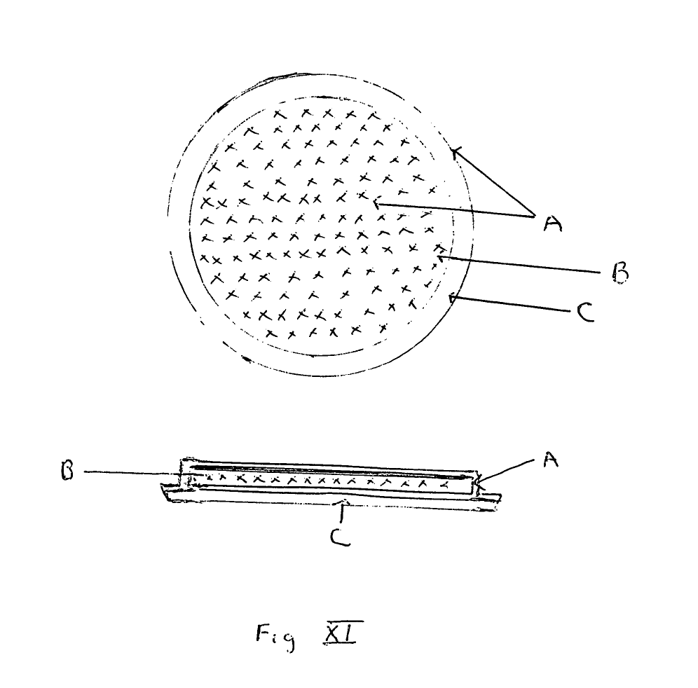

### FIELD OF INVENTION

This invention relates to methods and apparatuses for providing treatment or promoting health through the application of resonant electromagnetic radiation to the body in order to balance acupuncture points. The measurable resonant frequencies of substances that have the ability to balance acupuncture points have been cataloged in electronic devices in prior art. The resonant signals derived from these substances have been duplicated and stored in these devices so that they may be applied to a subject that wishes to balance acupuncture points. It is my idea and invention to use a holding device with these substances encased in a waterproof compartment allowing these resonant signals to balance the acupuncture points without the use of needles or electronic devices.

### SUMMARY OF INVENTION

The invention relates to a method and apparatus for holding substances on the body that have been found to balance acupuncture points. The apparatus is a holding device which may be a patch, locket, jewelry, belt or may be incorporated into clothing. The holding device has a waterproof non transdermal compartment that contains these particular substances. This substance may be a natural or manufactured substance such as a food, a chemical, animal dander, pollen, a pharmaceutical drug, homeopathic remedy, herb, vitamin, mineral or a biological organism. From this list of substances, the inventor of U.S. Pat. No. 6,142,927 has observed 40,000 different substances that produce resonant frequencies that have a balancing effect on acupuncture points. They are selected by their ability to send out resonant signals that have been proven to balance acupuncture points. If a substance is found to balance the electrical resistance across an acupuncture point then it may be worn by a subject to be afforded the therapeutic effect of continually balanced acupuncture points. It has been noted that if an operator measures the resistance at acupuncture point and then places a substance at or near the subject, with all other variables remaining the same, and repeats the measurement and the resistance becomes balanced then the substance may be considered a source of balancing.

As stated above the inventor of U.S. Pat. No. 6,142,927 has produced 40,000.00 substances that produce resonant frequencies that are able to balance acupuncture points. In this patent he then replicates these resonant signals in an electronic device that he has patented. As stated in his patent these product signals that normalize acupuncture points are taken from products that may be vitamins, herbs, minerals, foods (nutrients including amino acids), homeopathic remedies, herbs, animal dander, chemicals, pharmaceutical drug, pollen or biological organisms. My invention eliminates the use of this electronic device. Instead the acupuncture points are effected by holding these same substances next to the body so that the resonant signals can directly balance the acupuncture points.

Therefore, by using appropriate substances from the list above that have been found to balance acupuncture points, therapy can be administered continually by wearing such a holding device. From this list of substances are nutrients (including amino acids). This fact is pertinent to the following observed example of the utility of this invention.

According to The Journal of Chinese Medicine, no. 94, October 2010, (Immediate Effect of Acupuncture on Strength and Performance, a randomized, control crossover trial, European Journal of Applied Physiology) balancing of acupuncture points shows an increase in muscle strength. Also, in The Journal of Strength and Conditioning Research 24 (5) 1421-1427 2010 reviewed studies show the use of acupuncture in resistance and endurance sports activities demonstrates increase muscle strength and power.

Increased muscle strength by the topical placement of nutrients including amino acids in a non transdermal holding device has been claimed by my U.S. patent application Ser. No. 10/302,527 entitled Method for Increasing Muscle Strength. Subsequently, the topical placement of amino acids in a non transdermal apparatus to increase muscle strength has been claimed by U.S. patent application Ser. No. 12/915,419 entitled Biomolecular Wearable Apparatus. Both application use similar bench press trials on athletes to demonstrate this utility.

Therefore, nutrients (including amino acids) cause normalization of acupuncture points. This normalization of acupuncture points has been associated with increased muscle strength. The topical placement of amino acids and nutrients has been demonstrated to improve muscle strength in two independent double blind studies from these two applications cited above.

The device and method thus offers therapy on the body that will increase muscle strength. While this is an example of the method and apparatus, it is by no means the only ailment or problem that can be treated by balancing acupuncture points using this method and apparatus. In fact, this applicant does not identify any limits or problems for which the human body may be treated by this method and apparatus, the utilization of the present invention is only limited by the resonant signals that are produced by the particular substance or substances in the holding device that have the ability to balance acupuncture points.

In conclusion, the invention relates to an apparatus and method which comprises a holding device containing a waterproof compartment containing substances that give off natural frequencies that normalize acupuncture points. Therefore by placement of the said device on the surface of the body the utility of the invention may be realized. The utility of the invention is the balancing of acupuncture points that is afforded continually by wearing said holding device. The advantage of this invention over prior art is that it does not utilize needles or electrical devices which are cumbersome, uncomfortable and require the need to go to a health care professional to receive therapy. In addition the therapy may be afforded continually while wearing the holding device.

### DETAILED DESCRIPTION OF INVENTION

The invention utilizes the ability of substances to send out resonant frequencies that have the ability to balance acupuncture points. This ability of these resonant frequencies to affect resistance readings across acupuncture points has been measured using an ohmmeter. In U.S. patent #'927, the method of finding substances that can resonate with acupuncture points and have a balancing effect on these points is outlined. My invention simply takes these substances, places them in a non transdermal holding device, and this results in balanced acupuncture points while wearing the device. The holding device must only secure the resonant producing material to the surface of the body. Specific locations on the body are not required since according to '927 the substance has only to be brought near the body to balance acupuncture points. The method of securing these resonant producing materials on the body will become clearer after the following description of some of the various holding devices possible. The various types of holding devices that together with the active principle (substance which gives off resonant frequencies that can balance acupuncture points) make up the invention are illustrated for the purpose of illustration and without restriction.

FIG. I A. This illustrates a hat. The holding device is incorporated in a band (1) that goes around the circumference of the hat holding the active principle next to the skull.

FIG. I B. This is a longitudinal cross-section of the band showing the waterproof housing (2) which makes up the compartment holding the active principle.

FIG. II A. This pictures the holding device as a locket or disc. This AP view shows the outer plastic covering of the device (3), a clasp (2), and chain (3) which allows the device to hang around the neck of the subject at locations on the chest or solar plexus.

FIG. II B. This pictures a cross sectional view of the locket illustrating the plastic membrane (4) and the resulting waterproof compartment containing the active principle.

FIG. III A. This illustrates an AP view of a pedal device which may be a shoe, a pedal insert, or the base of a sandal. The dotted outline indicates the position of the active principle which is placed between a. fabric, leather material or some other appropriate material (1) that pedal inserts could be made of.

FIG. III B. This illustrates a longitudinal cross section of this pedal device. The outer covering (2) of the device may be composed of leather, plastic, or rubber making a waterproof housing (3) which contains the active principle (4).

FIG. IV A. This pictures the holding device as a belt. This AP view shows the outer covering (1) of the belt which is preferably leather, but may be plastic or other material. The dotted line indicates the location of the active principle which would be held next to the waist in this particular device.

FIG. IV B. This is a longitudinal cross section of the belt illustrating the outer covering (1) that surrounds a waterproof compartment that may be in the form of a plastic insert (2) which surrounds the active principle (3).

FIG. V A. This pictures an AP view of a holing device in the form of veterinary apparel. In this case a horse blanket is illustrated. The dotted line indicates the area where the active principle is located which is between the outer fabric and the under surface of the horse blanket.

FIG. V. B. This is a longitudinal cross sectional view of the horse blanket as a holding device. The outer fabric (2) of the device surrounds the waterproof housing (3) which houses the active principle (4).

FIG. VIA. This depicts a drawing of beads or stainless steel or ceramic balls which are 20× the actual size. The balls are hollow and constructed of waterproof material that can compose a holding device.

FIG. VIB. This pictures a magnified cross section of one of these hollow balls illustrating the waterproof outer shell (1) and the compartment (2) which contains the active principle.

FIG. VI C. When these balls are mixed with dental cement or amalgam, they can form a holding device that can be incorporated into a filling affording the envisioned therapeutic effect continually. Thus an ordinary filing is transformed into a therapeutic device.

FIG. VII A This pictures a mattress (1) that has the active agent sandwiched between two layers of fabric.

FIG. VII B . This pictures a longitudinal cross section of the mattress. A waterproof compartment (3) lies under the fabric of the mattress (2). This compartment (3) contains the active principle (4) which can supply the envisioned therapeutic effect while resting.

FIG. VIII A. This depicts a hollow spool of thread. In the magnified view (1) of the thread, the thread is shown to be hollow allowing the active principle to be incorporated into the resulting waterproof chamber. The resulting fiber can be woven into various articles of clothing that would provide the envisioned therapeutic effect while being worn.

FIG. VIII C. This illustrates the various articles of clothing when such fibers are used in producing clothing. Specifically, a shirt (6), gloves (7), pants (8), and socks (9) are illustrated.

FIG. IX. A. This illustrates a watchband as a holding device. The active principle is sandwiched between two pieces of leather which make up the watchband.

FIG. IX B. This illustrates a cross section of the watch band. The leather material (1) surrounds a waterproof plastic insert (2) which contains the active principle (3).

FIG. X A This pictures an AP view of a ring that can act as a holding device. It may be composed of a metal such as gold, silver, platinum, copper, etc.

FIG. X B As seen in this cross sectional view, the hollow chamber of the ring can act as a holding device for the active principle thus providing the envisioned therapeutic response while being worn.

FIG. XI. A. This illustrates the holding device as a non transdermal patch. In this AP view the plastic covering (A) forms a compartment (B) containing the active principle. The patch is held on various locations on the body with a medical adhesive (C).

FIG. XI. B. This pictures a cross sectional view of the non transdermal patch. The plastic lining (A) forms a waterproof compartment (B) containing the active principle. A medical adhesive (C) is used to secure the patch to the skin.

1. A method of balancing acupuncture points by wearing a non transdermal holding device that incorporates a waterproof housing that contains a least one substance that sends out resonant frequencies that can balance acupuncture points.

2. A method of balancing acupuncture points according to claim 1 where said substance that gives off resonant signals that balance acupuncture points is itself a waterproof substance such as a metal or polarized film that can be put into a holding device without the need of waterproof housing to make it non transdermal.

3. A method of balancing acupuncture points according to claim 1 that affords the many health benefits of balanced acupuncture points without the use of needles or electrical devices.

4. A method according to claim 1 of balancing acupuncture points continually by wearing such a holding device.

5. A method according to claim 1 where said substance can be a manufactured substance or anything found in nature and is only limited by its ability to send out resonant frequencies that balance acupuncture points.

6. A method according to claim 1 where said substance sends out resonant frequencies that balance acupuncture points and thereby increase muscle strength.

7. A method according to claim f where said holding device is a non transdermal patch that contains amino acids that sends out resonant signals that balance acupuncture points.

8. A method according to claim one where the holing device is in the form of a band around a hat.

9. A method according to claim one where said holding device is in the form of a locket or jewelry.

10. A method according to claim one where said holding device is in the form of a pedal insert of a shoe.

11. A method according to claim one where said substance that balances acupuncture points may be a nutrient, vitamin, amino acid, homeopathic remedy, medicament, or mineral.

12. A method according to claim 1 where said substance that gives off resonant frequencies that balance acupuncture points may be in the form of veterinary apparel that can afford the benefits of balanced acupuncture points to animals.

13. A method according to claim one where said holding device is in a blanket so that the envisioned therapeutic effect may be realized while sleeping.

14. A method according to claim one where the said holing device can be incorporated into the amalgam of filling thus producing a resonant producing implant that can balance acupuncture points.

15. A method according to claim one where the substance producing resonant signals that balance acupuncture points has been incorporated into the hollow waterproof weave of clothing that may be worn by a subject that desires balanced acupuncture points.
